(19) United States
(12) Patent Application Publication
(10) Pub. No.: US 2008/0293133 A1
(43) Pub. Date: Nov. 27, 2008

(54) Textiles for use in bioreactors for expansion and maintenance of cells

(75) Inventors: Lola M. Reid, Chapel Hill, NC (US); Bhupender S. Gupta, Cary, NC (US); Jeffrey M. MacDonald, Chapel Hill, NC (US)

Correspondence Address: FOLEY AND LARDNER LLP;SUITE 500, 3000 K STREET NW, WASHINGTON, DC, 20007, US

(73) Assignees: North Carolina State University; The University of North Carolina at Chapel Hill

(21) Appl. No.: 12/153,170

(22) Filed: May 14, 2008

Related U.S. Application Data
(63) Continuation of application No. 10/697,870, filed on Oct. 31, 2003, now abandoned.
(60) Provisional application No. 60/422,512, filed on Oct. 31, 2002.

(51) Int. Cl. C12M 3/00; C12M 1/00

(52) U.S. Cl. 435/294.1; 435/307.1

(57) ABSTRACT

A bioreactor for three-dimensional culture of liver cells is disclosed. The device is characterized by the use of textile vasculatures. A model and method for optimizing vasculature parameters is also disclosed. Liver acinar structure and physiological parameters are mimicked by sandwiching cells in the space between the two innermost woven textile hollow fibers, and creating radial flow of media from an outer compartment, through the cell mass compartment, and to an inner compartment. The theoretical optimum hydraulic permeability for the two innermost semi-permeable membranes is determined based on physiological hepatic sinusoidal blood flow and pressures. Experimental studies using a flow rate and pressure monitoring systems in conjunction with phase-contrast velocity-encoded MRI confirm theoretical results. Novel woven vascular tubes with optimum hydraulic permeability are disclosed for culturing hepatocytes in the multi-coaxial bioreactor.

### CROSS REFERENCE TO RELATED APPLICATIONS

This application is a Continuation of U.S. Utility Patent Application Ser. No. 10/697,870, filed Oct. 31, 2003, and is incorporated herein by reference.

### FIELD OF THE INVENTION

The present invention relates generally to devices and processes of making and using devices for cell culture. In particular, the present invention relates to devices and processes of making and using devices for growth or maintenance of eukaryotic cells.

### BACKGROUND

Artificial organs, which are devices made entirely of non-biological materials, have greatly advanced health care. Artificial organs and tissue substitutes, including kidney dialysis machines, mechanical respirators, cardiac pacemakers, and mechanical heart pumps have sustained many people with desperate life-threatening diseases. The utility of such artificial organs is reflected in their widespread use.

Bioartificial organs are artificial organs designed to contain and sustain a viable biological component. Many biological functions are even more complex than simply generating a voltage potential at regular intervals, as occurs in the simplest of pacemakers. Examples include biosynthesis of blood components and catabolic processing of deleterious agents. The liver, endocrine glands, bone marrow, and kidney are prominent in such specialized biochemical functions. Artificial organs without a biological component cannot reproduce the complex biochemical functions executed by these organs.

The artificial kidney, sometimes termed the kidney dialysis machine, for example, serve admirably as substitutes for their biological analogs. Kidney dialysis machines illustrate both the benefits and shortcomings of purely artificial organs. Kidney dialysis machines effectively remove urea, creatinine, water, and excess salts from the blood, thus partly fulfilling major roles of the natural kidney. Artificial kidneys have postponed deaths of patients in renal failure. However, kidney dialysis machines are insufficiently selective and inappropriately remove biological components, such as steroid hormones, that a functioning natural kidney does not. Consequently, dialysis over an extended period may result in bone loss, clotting irregularities, immunodeficiencies, and sterility. Thus, considering the artificial kidney as a model, the capacity of artificial organs to mimic biologic functions is limited and may result in adverse implications for the patient under treatment.

Liver failure is classified into several major types, including acute liver failure, chronic liver disease, and multiorgan failure. The main etiologies of liver failure are viral hepatitis and hepatotoxicity induced by drugs and toxins. Advanced liver failure results in encephalopathy and coma, and may be fatal. Treatment focuses on stabilizing the patient until spontaneous recovery of liver function, or until liver transplantation. In the aggregate, the annual mortality attributable to liver failure exceeds 27,000 annually in the United States.

A patient in hepatic failure, unlike a patient in renal failure, cannot be specifically treated because there is no hepatic equivalent to renal dialysis. Currently, the only available treatment for refractory liver failure is hepatic transplantation. Many patients in hepatic failure do not qualify for transplantation because of concomitant infection, or other organ failure. Because of organ shortages and long waiting lists, even those who qualify for liver transplantation often die while awaiting an allograft. UCLA reported that one quarter of their transplant candidates died before a liver could be obtained. Organs suitable for transplant in the pediatric age group are even more scarce (Busuttil, R. W. et al. Ann Surg 1987, 206, 387).

The natural liver has four major classes of biochemical functions. First, the liver biosynthesizes a wide range of proteins, including major acellular components of blood, such as serum albumin, alpha-anti-trypsin, alpha-macroglobulin, enzymes, clotting factors, carrier molecules for trace elements, and the apo-lipoproteins. The liver then releases these components to the blood circulation. The liver also maintains appropriate plasma concentrations of amino and fatty acids. Second, the liver has a major role in detoxification reactions. The liver oxidizes or conjugates many harmful external poisons, processes that usually, but not always, diminish the poisonous character of the toxins. The liver also destroys excess hemoglobin, metabolizes the porphyrin molecules of hemoglobin, and recycles the iron component. Third, waste products, such as bilirubin, are conjugated and excreted via the biliary tree. Fourth, the liver synthesizes and secretes the bile salts, which serve as detergents that promote the emulsification and digestion of lipids. The multiplicity and biochemical character of liver function vastly increase the complexity of extracorporeal hepatic support.

Historically, non-biologic artificial liver substitutes have depended on hemodialysis and hemoperfusion, but have been of very short-term and highly limited benefit (Abe, T. et al., Therapeutic Apheresis 2000, 4:26). In contrast to purely artificial organs, an effective liver replacement must have a biological component. The liver is the most massive organ in the human body, exclusive of distributed organs such as skin, gut, hematopoietic system, and vasculature. Sustaining a large mass of functioning liver cells in vitro presents a variety of hurdles. At least eight major problems to developing a functional bioartificial liver can be described: 1) growing or obtaining appropriate and viable cells; 2) providing for a critical minimum mass of cells; 3) supplying oxygen to the cells; 4) supplying nutrients to the cells, and removing cell waste products efficiently; 5) limiting shear forces and hydrostatic pressures, 6) inducing or sustaining a differentiated cell phenotype with the capacity for biosynthesis and biotransformation of toxins; 7) maintaining sterility; and 8) preventing liver tissue rejection or lysis by complement.

1) Growing or obtaining appropriate and viable cells. Liver cells for potential use in bioartificial livers can be established cell lines, primary isolates from human or animal livers, or primordial liver cells however, secretion of tumorigenic factors is negatively affecting FDA approval of BAL designs incorporating cell lines (Xu, A. S. L. et al., 2000 in Lineage Biology and Liver, Lanza, R. P., Langer R., and Vacanti, J. (Ed.), Academic Press, San Diego, pp. 559-597). Cell lines of liver are available, for example HepG2 and C3A, that express many functions of differentiated liver. Cell lines offer the potential of growing sufficient numbers of cells in an extracorporeal mass cell culture system, or bioreactor, for sustaining a patient because the growth of cell lines is not limited by cell senescence, but by nutrient availability. Primary human or animal liver cells can also be obtained in the numbers required for a functional bioartificial liver. However, the use of human liver for cell preparation is limited by its lack of availability, and the use of animal liver for cell preparation suffers from some degree of cellular incompatibility. Acute cellular incompatibility results from the binding of antibodies that recognize foreign cells followed by the binding of proteins of the complement system and lysis of the foreign cells. Longer-term cellular incompatibility mechanisms also exist, but should not present any problems for the use of bioreactors as interim or “bridge” medical products. A possible alternative to initial inoculation with a large mass of differentiated cells is the expansion of liver stem cells that are progenitors of mature liver cells. Recent reports suggest that liver progenitor cells go through multiple cell divisions on the path toward maturation and differentiation (Brill, S. et al., Differentiation 1995, 59, 95; Sigal S. H. et al., Differentiation 1995, 59, 35). Suitable control of the growth and differentiation processes with staged application of appropriate cytokines can permit preparation of a clinically useful quantity of cells.

2) Providing for a critical minimum mass of cells. The adult human liver has a mass of about 1400-1600 grams, and features a considerable reserve, or redundant, capacity. It is estimated that human survival can be sustained with about 15-20% of the total liver mass. The figure of 20% of the liver mass corresponds to about 5×10¹⁰ cells (Kasai et al. Artif Organs 1994, 18, 348). Most, if not all, previous bioartificial liver designs suffer from a woefully inadequate cell capacity. That is, such devices are capable of sustaining far fewer than 5×10¹⁰ cells, often orders of magnitude fewer cells. Without the cell mass critical for biosynthesis of plasma components and detoxification reactions, these other designs have little clinical utility.

3) Supplying oxygen to the cells. The functional units of most organs such as nephron, acinus, alveoli, microvilli, skin, etc. consists of a capillary bed across which is a physico-chemical gradient. These gradients are controlled by mass transfer effects. Oxygen is the primary nutrient that is limiting in cell cultures (Macdonald, J. M. et al. NMR Biomed 1998, 11, 1; Glacken M. W. et al. Ann NY Acad Sci 1983, 413, 355). ‘Integral’ oxygenation, or aeration inside the bioreactor containing the biological or chemical material of interest, greatly enhances mass transfer of oxygen and carbonic acid. The formation of the latter can be used to control pH.

Oxygen is generally the limiting nutrient in hollow fiber bioartificial livers (Catapano, G. et al. Int J Art Organs 1996, 19, 61) primarily because hepatocytes are highly aerobic cells which causes problems of oxygen mass transfer. Oxygen has a relatively high diffusion coefficient and its mass transfer from blood in the liver sinusoids to hepatocytes is dominated by diffusion rather than convection (i.e., convection and perfusion are caused by pressure gradients). These effects are because an oxygen molecule is much smaller than other nutrients such as a glucose molecule, or than biosynthetic products such as proteins, and because the hepatocytes generate steep concentration gradients in bioartifical livers. With known rates of oxygen diffusion and oxygen consumption, and reasonable estimates of cell density, the diffusion distance at which oxygen utilization becomes the rate-limiting factor for growth is approximately 200 μm (Macdonald, J. M. et al., 1999, in Cell Encapsulation Technology and Therapeutics, Kuhtreiber, W., Lanza, R. P. and Chick, W. L. (Eds.) Birkhauser Boston, Cambridge, pp. 252-286. In bioartificial livers with serial oxygenation aerated with air, oxygen becomes axially limiting in perfusion media by 25 mm (Macdonald et al., 1999, supra).

Hepatocytes have a high metabolic rate and require a continuous oxygen supply. The oxygen consumption rate ranges from 0.59 to 0.7 nmole/s/10⁶ cells for HepG2 cells (Smith, M. D. et al Int J Artif Organs 1996, 19, 36) and is 0.42 nmole/s/10⁶ cells for isolated hepatocytes (Rotem, A. et al. Biotech Bioeng 1992, 40, 1286). Integral oxygenation, that is, continuous supply of oxygen along the path of media supply to the cells, is essential to supplying oxygen to liver cells. Serial oxygenation, which is oxygenation at one or a few places in the fluid line of media supply cannot sustain the mass of liver cells needed for an effective bioartificial liver. A difficulty with serial oxygenation is that the solubility of oxygen in aqueous media unsupplemented with oxygen carriers is so low that any oxygen present is quickly depleted by cell metabolism. In fact, in longitudinal flow along a conventional bioreactor semipermeable membrane, hepatocytes deplete oxygen within 2.5 centimeters along the path and therefore convective oxygen mass transfer via increasing Starling flow is improved. Increasing flow rates through conventional bioreactors can cause fiber breeches and adversely affect hepatocyte function (Callies, R. et al., Bio/Technology 1994 12:75). Thus, bioartificial liver designs that do not provide for adequate oxygen delivery are able to support only a limited number of cells. In addition, the flux of oxygen in a diffusion-limited system constrains cells to grow very near (less than about 0.2 mm) to the supply of oxygen. For example, U.S. Pat. No. 5,622,857 to Goffe discloses a bioreactor with some coaxial and some parallel semi-permeable hollow fibers. The Goffe design allows integral oxygenation but does not constrain the thickness of the cell compartment. The fiber-to-fiber spacing in that design is 3-5 mm so that there is not strict control of the oxygen diffusion distance. Similarly, U.S. Pat. No. 5,183,566 to Darnell et al. discloses a bioreactor with bundles of hollow fibers in parallel. The Darnell et al. design does not permit a multitude of individual multi-coaxial fiber bundles to be built-up with accurate and reproducible diffusion distances, and the design is not easily scaled-up. The Darnell et al. design uses bundles of parallel fibers, again not effectively addressing the issue of oxygen diffusion.

4. Supplying nutrients to the cells, and removing cell waste products efficiently. The issue of supplying nutrients such as carbohydrates, lipids, minerals, and vitamins has been successfully solved by several variants of hollow fiber technology, and these features must be successfully incorporated into any viable bioartificial liver or bioartificial organ design. Similarly, the issue of removing metabolic wastes is usually handled by the same system that supplies the nutrients. The consumption rates for glutamate, pyruvate, and glucose are typically in the range of 0.03 to 0.3 nmol/S/10⁶ cells, with reasonable assumptions for cell density and growth rate (Cremmer, T. et al. J Cell Physiol 1981, 106, 99; Imamura, T. et al. Anal Biochem 1982, 124, 353; Glacken, M. Dissertation 1987). The diffusion rates of oxygen in tissue are similar to those of pyruvate in water, and higher than those of glucose. As these consumption rates are less than the oxygen consumption rate, oxygen is the limiting nutrient in most conditions.

5. Limiting shear forces and hydrostatic pressure. For a given bioreactor there is an optimum balance of convection and diffusion for adequate oxygen mass transfer without creation of severe oxygen gradients. For example, using a nontoxic oxygen range, <0.4 mM (solubility constant is 1.06 mM/atm, for air solubility is 0.2 mM at 37° C.), the convective component of oxygen mass transfer should be increased as cells are increasingly farther than 0.2 mm from supply of oxygen (Macdonald et al., 1999, supra.). Although the partial oxygen tension in the liver sinusoid is about 70 mm Hg near the portal triad dropping to 20 mm Hg near the central vein, which equates to a range of 0.096 to 0.027 mM of free oxygen, the hemoglobin-bound oxygen ranges from 6.26 to 2.91 mM. The velocity of blood flow in the liver sinusoid is about 0.02 cm/s while the oxygen diffusion coefficient is about 4 orders-of-magnitude less, or 2×10⁻⁶ cm²/s. However, hepatic function is adversely affected with increasing shear forces, and in vivo hepatocytes are protected by a layer of endothelia and extracellular matrix in the space of Disse. Sufficient shear forces will kill hepatocytes. Others have found that shear forces induce specific cytochrome P450's (Mufti N. A. and Shuler, M. L., Biotechnol. Prog., 1995, 11, 659). A recent study has shown that liver regenerates faster with 90% than with 70% hepatectomy and this was attributed to greater shear forces (Sato, Y. et al., Surg. Today, 1997, 27, 518). However, this faster regeneration could also be due to enhanced oxygen, nutrient, and agonist mass transfer. Therefore, there is some maximum level of shear force that hepatocytes can sustain while still displaying optimal function. This maximum level can be increased if a layer of endothelia protects hepatocytes.

To increase convection, hydrostatic pressure gradients are increased. Elevated hydrostatic pressures can implode hepatocytes. Therefore, it is important to stay below these pressures. It is possible to cause 100% mortality of isolated rat hepatocytes by generating hydrostatic pressures of greater than 7 psi (>300 mm Hg) for longer than 2 minutes while inoculating these cells into coaxial bioreactor using a syringe.

6) Inducing or sustaining a differentiated cell phenotype with the capacity for biosynthesis and biotransformation of toxins. The use of the differentiated phenotype of liver cells is necessary to produce a useful bioartificial liver because the specialized functions of the liver, including biosynthesis of blood components and detoxification of toxins, are associated with the differentiated phenotype. These specialized functions are lost in whole, or in part, as the cells dedifferentiate, which often happens in isolated primary cell culture. In contrast, the form of liver cells capable of rapid growth is the dedifferentiated phenotype, leaving the practitioner to balance two opposing needs (Enat, R. et al. Proc Natl Acad Sci USA, 1984, 81, 1411 ). Some reports suggest that the phenotype of liver cells may be modulated by the presence of cytokines and extracellular matrix components. In particular, the extracellular matrix components rich in collagen IV and laminin, produced by the Engelbrech-Holm Sarcoma (EHS) cells and available commercially as MATRIGEL™, when used with hormonally defined media induces a differentiated phenotype (Enat, R. et al., supra; Bissell, D. M. Scan J Gasterenterol-Suppl 1988, 151,1; Brill, S. et al. Proc Soc Exp Biol Med 1993, 204, 261 ).

7) Maintaining sterility. The implementation of facile sterilization procedures for bioreactors and associated components is essential for clinical utility of extracorporeal bioartificial organs. Fortunately, the procedures for sterilization are well established, including standard methods both for sterilization of extracorporeal devices and for maintaining asepsis by standard in-line filters.

8) Preventing liver tissue rejection or lysis by complement. Rejection of foreign tissue can occur by a rapid process known as complement-mediated lysis that involves binding of circulating antibodies to the foreign cell surface, attachment of the proteins of the complement system, and lysis of the offending cell. The cell-mediated immune system is responsible for delayed rejection reactions. However, the cell-mediated immune system should not play a major role in bioreactor systems that do not permit direct contact of host and donor cells. Foreign body reactions, for example, against the structural components of bioreactors, are also cell-mediated and should therefore not constitute substantial obstacles.

Examples of current bioreactors used for expansion and/or maintenance of cells include those that make use of hollow fiber bioreactors, flatbed bioreactors, flatbed microchannel bioreactors, and roller bottles.

Hollow fiber bioreactors incorporate hollow fibers that are extruded hollow tubes and prepared from polypropylene, polysulfone, polyamide, regenerated cellulose, and other extrudable polymers. These hollow fibers do not have adequate permeability to allow long-term survival and functioning of cells in the bioreactor.

Flatbed bioreactors use impervious, rigid surfaces such as glass or culture plastic as a surface for cells. The mass transfer of nutrients is achieved by flow of the media directly across the cells. These bioreactors are unable to achieve the requisite mass of cells needed for clinical use or for some tissue-specific functions. Moreover, the rigid and impervious surfaces used block requisite three-dimensional shape changes essential for cells to express tissue-specific functions.

Flatbed microchannel bioreactors use cells sandwiched in extracellular matrix and between two plates of rigid, impervious surfaces such as glass or culture plastic. These bioreactors are incapable of achieving the requisite mass needed for clinically useful bioreactors and are difficult to use for most experimental studies.

Roller bottles consist of glass or plastic bottles in which cells are expanded and/or maintained on the inner surface of the bottles. The cells are grown as monolayers on the surface of the bottles making the achieving of high density cell populations dependent upon the surface area of the inner surface of the bottles. Also, the cells are blocked in achieving three dimensional shapes requisite for optimal expression of tissue-specific functions.

It would be desirable to enable the cells to expand to high densities or be inoculated in the bioreactors at high densities to yield very high density, three-dimensional cultures and yet be able to survive long-term (weeks to months theoretically) by providing the supply lines, the hollow fibrous structures, with the needed permeability for mass transfer of nutrients, gases, and wastes. To this end, Applicants disclose herein a use of optimized medical textile products.

From the first appearance more that 4000 years ago to their present use in products ranging from gowns and wound dressings to arterial and skin grafts, fibers and fabrics have been explored as potential materials for applications in medicine and surgery. This continuing interest has its basis in the unique properties of fibers—which in many respects resemble biological materials—and in their ability to be converted into a wide array of desired end products.

Medical textile products are based on fabrics of which there are four types: woven, knitted, braided, and nonwoven. The first three of these are made from yarns, whereas the fourth can be generated directly from fibers, or even polymers. There is, therefore, a hierarchy of structure. The performance of the final textile product is affected by the properties of the polymer whose contribution in the final product is modified by the structure at two to four different levels of organization.

Textile medical products are made from biocompatible polymers. Biocompatibility, or the reactivity of body tissues and fluids when in contact with polymeric structures, is governed both by chemical and physical characteristics of polymers (See for example, Gupta, “Medical Textile Structures: An Overview,” Medical Plastics and Biomaterials, 5 (1): 16-30 (1998) incorporated herein by reference in its entirety). Absorbable materials (e.g. polyglactin, polyglycolic acid, polyglyconate) typically excite greater tissue reaction whereas semiabsorbable materials (e.g. cotton, silk) cause less reaction. Non-absorbable materials (e.g. polyester, nylon, polypropylene, polytetraflouroethylene, polyurethane) tend to be inert and relatively the most biocompatible. Polymers are extruded to make monofilament fibers, which are converted to yarns by twisting or entangling processes that improve strength, abrasion resistance, and handling. Nonwoven fabrics are made directly from fibers or polymers, creating high bulk absorbent and usually isotropic fabrics. These are used in numerous medical applications (wipes, sponges, dressings, gowns) and, with proper polymer base, as biodegradable scaffolds in tissue engineering of liver implants (see for example, Mooney, et al, “Long-term Engraftment of Hepatocytes Transplanted on Biodegradable Polymer Sponges,” J. Biomed. Mater. Re., 37: 413-420 (1997) incorporated herein by reference in its entirety). Weaving, knitting, or braiding of yarns make highly organized anisotropic fabrics that are suited for many implants.

Fabrics that are woven are usually dimensionally highly stable but less extensible and porous than are the knitted or the braided structures. One disadvantage of wovens is their tendency to unravel at the edges when cut squarely or obliquely for implantation. However, the stitching technique known as a Leno weave—in which two warp threads twist around a weft—can be used that substantially alleviates this fraying or unraveling problem (See for example, Kapadia et al., “Woven Vascular Grafts.” U.S. Pat. No. 4,816,028 (1989) incorporated herein by reference in its entirety). The primary problems with knits are that they are dimensionally unstable and their porosity is difficult to control and engineer. Braiding technology can be used to produce a flat or a cylindrical structure; however, it does not easily lend to producing a stable hollow tube. Some of the current research in the biomedical field is focused on the use of absorbable and elastomeric yarns or fibers into woven materials, and the use of coatings such as albumin (See for example, Mehri, et. al., “Cellular Reactions to Polyester Arterial Prostheses Impregnated with Cross-Linked Albumin: In Vivo studies in Mice,” Biomat. 10(1): 56-58 (1989)), gelatin (Bordenave et al., 1989), and collagen (Frey, et al., “Prosthetic Implants,” U.S. Pat. No. 5,176,708 (1993) each incorporated herein by reference in its respective entirety).

The ideal artificial vasculature is one that is biocompatible, has the desired porosity and the required mechanical patency (i.e., the ability to resist permanent change in physical size, shape, structure, and properties).

Specifically, a bioreactor that permits cells to survive and function indefinitely is needed. Preferably this bioreactor enables cells to expand to high densities or be inoculated in the bioreactors at high densities to yield very high density, three-dimensional cultures and yet be able to survive long-term (weeks to months theoretically) by having the needed permeability for mass transfer of nutrients, gases, and wastes. Such a bioreactor is disclosed herein.

### SUMMARY OF THE INVENTION

One aspect of the present invention is to provide varying embodiments of an apparatus which provides efficient oxygen delivery to large masses of cells in a bioreactor cell culture and transfer of beneficial biosynthetic cell products to the patient, and methods of use therefor, comprising multi-coaxial hollow fibrous structures assembled from woven textile fibers with a porosity that is governed by the weave design. Woven textile vasculature may be used to make hollow fibrous structures in hollow fiber bioreactors, as a cell surface for flatbed bioreactors, or in bags for three-dimensional culture systems for expanding and maintaining cells. The woven textile vasculature can be prepared from any fiber or combinations of fiber chemistries such as polyester, cotton (or other forms of cellulose), biodegradable fibers, etc. and with any weave design desired. The weave design and the chemistry of the fibers can be adjusted to provide the requisite permeability of the hollow fibrous structures for engineering of tissues.

A further aspect of the present invention is to provide an apparatus which permits cells to be contained in a thin annular space adjacent to continuously oxygenated and flowing nutrient medium that provides essential oxygen and nutrients and carries away metabolic products.

A further aspect of the present invention is to provide an apparatus for the collection of the biosynthetic products of large masses of cells in a bioreactor.

A further aspect of the present invention is to provide an apparatus to detoxify blood or plasma from a patient unable to remove or inactivate these toxins.

A further aspect of the present invention is to provide an apparatus to serve as a substitute liver.

A further aspect of the present invention is to provide varying embodiments of an apparatus which provides efficient oxygen delivery to large masses of cells in a bioreactor cell culture and transfer of beneficial biosynthetic cell products to the patient, and methods of use therefor.

A further aspect of the present invention is to provide vasculatures in the quality and the quantity ideally suited for the success of bioartifical livers.

A further aspect of the present invention is to provide bioreactors for use in academic and industrial research on cells.

A further aspect of the present invention is to provide a means for expansion of cells to high densities for use in biochemical/cell/molecular studies in research or clinical programs (e.g. cell therapies, gene therapies).

A further aspect of the present invention is to provide protein manufacturing in cells maintained in bioreactors.

A further aspect of the present invention is to provide organ assist devices (e.g. liver assist devices) to support patients with failing organs.

A further aspect of the present invention is to provide implantable tissues created ex vivo in woven tubes or woven bags prepared with biodegradable fiber chemistries.

A further aspect of the present invention is to provide vasculatures in a number of sizes and structures with properties ideally suited for maintaining and expanding cells in bioreactors.

A further aspect of the present invention is to provide biodegradable vasculatures in which a biodegradable polymeric fiber (such as polylactide) is used along with non-biodegradable material (such as polyester) in the proportion that sets the upper and lower limits of porosity and the transition from one to the other takes place at the desired rate.

A further aspect of the present invention is to provide elastomeric vasculatures that distend to the required amount in the transverse direction.

A further aspect of the present invention is to provide such structure for transport of fluid. A further aspect of the present invention is to provide grafts in size and properties suited for by-pass use which are compatible with the transverse elongations of body arteries; e.g. elongations of the level of 20-30%.

A further aspect of the present invention is to provide these characteristics through the use of elastomeric threads, or threads containing a blend of regular and elastomeric materials, as the weft yarns for construction of vasculatures.

The bioreactor of the present invention, when used as a bioartificial liver, has a modular design to allow an easy adjustment in liver functional capacity depending on the weight of the patient, whether that patient is child, man, or woman, and on the degree of remaining liver function in the patient. The bioreactor of the present invention further has both plasma and nutrient medium compartments to permit the biotransformation of toxins in the patient plasma and to enhance the effective transfer of biosynthetic products from the bioartificial liver to the patient. When used with liver or other cells, this invention is useful in the preparation of biosynthetic products for patients, in experimental use, and use as a supplemental biotransformation apparatus for detoxification of blood. The toxins in the blood can include, but are in no way limited to, metabolic wastes, products of cell or erythrocyte break-down, overdoses of ethical pharmacologic agents such as acetaminophen, and overdoses of illicit pharmacologic agents. Ease of manufacture of the invention enables cost-effective commercial development.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other aspects of the invention, along with the various features of novelty, which characterize the invention, are pointed out with particularity in the claims annexed to and forming a part of this disclosure. For a better understanding of the invention, its operating advantages and the specific aspects attained by its uses, reference should be had to the accompanying drawings and descriptive matter in which there is illustrated preferred embodiments of the invention.

### BRIEF DESCRIPTION OF THE DRAWINGS

FIG. 1 illustrates woven vasculatures shown in flat and cylindrical forms.

FIG. 2 illustrates two means by which woven cylindrical tubes can be incorporated into the multicoaxialbioreactor, with the air chamber in the inner-most or outer-most compartments.

FIG. 3 illustrates the variables used in the implementation of Darcy's law.

FIG. 4 illustrates a liver lineage model.

FIG. 5 illustrates a multicoaxial bioreactor design.

FIG. 6 illustrates porous, biocompatible, biodegradable PLGA microcarriers for cells in bioreactors.

FIG. 7 illustrates physical analysis of the liver acinus.

FIG. 8 illustrates membrane fouling studies.

FIG. 9 illustrates the effect of no hemoglobin on oxygen mass transfer.

FIG. 10 illustrates a comparison of conventional with multicoaxial bioreactor.

FIG. 11 illustrates a hydrodynamic model.

FIG. 12 illustrates the use of MRI to determine axial flow.

FIG. 13 illustrates predicted pressure profile and optimum K₁ and K₂.

FIG. 14 illustrates membrane fouling and its adverse effect on mass transfer.

FIG. 15 illustrates dead-end and cross flow configurations for the fouling study.

FIG. 16 illustrates results of dead-end and cross flow configurations for fouling study.

FIG. 17 illustrates results of dead-end and cross flow configurations for fouling study.

FIG. 18 illustrates fouling studies of woven vasculature incorporated into multicoaxial bioreactors.

### DETAILED DESCRIPTION OF THE INVENTION

### Definitions

Annular space. The radial distance separating two adjacent vasculatures.

BAL. Bioartificial liver. Also, specific embodiments of the present invention: the scaled-up multi-coaxial vasculature bioreactor, the tight packed hollow vasculature bioreactor or the serially-linked bioreactor with a complement of liver cells, nutrient medium, and gases.

Bioreactor module. Coaxially-arranged semipermeable hollow vasculatures. One module forms the core of the multi-coaxial hollow vasculature bioreactor whereas the scaled-up multi-coaxial hollow vasculature bioreactor comprises many modules.

Biotransformation. The metabolic detoxification of blood or plasma by tissues or cells.

Fourth compartment. The compartment, if present, in a bioreactor embodiment that is bounded by the outside of the third hollow vasculature and the inside of the fourth, that is, adjacent, hollow vasculature, and is connected to two ports, the fourth compartment inlet port and the fourth compartment outlet port.

First compartment. The compartment in any of the bioreactor embodiments that is bounded in part by the inside of the first and innermost coaxial hollow vasculature and is connected to two ports, the first compartment inlet port and the first compartment outlet port.

Integral aeration. Exposure to a gas, typically air or oxygen with carbon dioxide, at almost all points along a flow path. Integral aeration is distinguished from serial aeration, in which a bubbler or gas exchange device is inserted at one point in the fluid circuit.

Manifold. A part of the bioreactor located at an end of the fibers and intended to physically separate compartments and split flow of fluids.

Microvasculature or microbore hollow fiber. A semipermeable hollow vasculature of 200 to 500 micrometer o.d.

Multi-coaxial hollow vasculature bioreactor. The bioreactor comprising three or more coaxially-arranged semi-permeable hollow vasculatures encased by a hollow housing.

Nutrient medium. The balanced electrolyte solutions enriched with sugars, trace minerals, vitamins, and growth enhancers. Each particular formulation is named by or for the formulator, sometimes with whimsical or non-illuminating designations. Nutrient media include, but are not limited to: RPMI 1640 (Roswell Park Memorial Institute, formulation #1640), Ham's F-12 (the twelfth formulation by Dr. Ham in his F series), DMEM (Dulbecco's modified Eagle's medium), and CMRL-1415 (Connaught Medical Research Laboratory formulation #1415). Nutrient media are routinely enhanced by addition of hormones, minerals, and factors known to those of ordinary skill in the art, including, but in no way limited to, insulin, selenium, transferrin, serum, and plasma.

One-sided multi-coaxial hollow vasculature bioreactor. The version of the multi-coaxial hollow vasculature bioreactor that has both inlet and outlet ports on the same end plate. This version is particularly adapted to NMR studies and to studies where access to all ports from one side is necessary.

Outermost compartment. The compartment in any of the bioreactors that is bounded by the outside of the outermost hollow vasculatures and the inside of the housing, and is connected to two ports, the outermost compartment inlet port and the outermost compartment outlet port.

Scaled-up multi-coaxial hollow vasculature bioreactor. The bioreactor comprising arrays of from about 20 modules to about 400 modules of coaxially-arranged semi-permeable hollow vasculatures, where the entire set of modules is encased by a hollow housing.

Second compartment. The compartment in a bioreactor embodiment that is bounded by the outside of the first and innermost coaxial hollow vasculature and the inside of the second, that is, adjacent, coaxial hollow vasculature, and is connected to two ports, the second compartment inlet port and the second compartment outlet port. In the one-sided multi-coaxial hollow vasculature bioreactor and in some dead-ended vasculature designs only one port provides access to the second compartment.

Serially-linked bioreactor. The system comprising a plurality of scaled-up multi-coaxial hollow vasculature bioreactors or of tight-packed hollow vasculature bioreactors, or a combination, in which two or more compartments are connected in a continuous and serial manner. In this context, each scaled-up bioreactor is referred to as a bioreactor subunit.

Third compartment. The compartment in any of the bioreactor embodiments that is bounded by the outside of the second hollow fiber and the inside of the third, that is, adjacent, coaxial hollow vasculature, and is connected to two ports, the third compartment inlet port and the third compartment outlet port.

Tight-packed hollow fiber bioreactor. The scaled-up bioreactor comprising arrays of from about 20 modules to about 400 modules of coaxially-arranged semi-permeable hollow vasculatures. Microvasculatures for aeration are arranged parallel and adjacent to the modules and the whole encased by a hollow housing.

Vasculatures. Vascular tubes made from woven fabric.

### Vasculatures

Ideally, cells should be expanded and maintained in three-dimensional systems such as bioreactors. In a preferred embodiment, the cells behave as closely as is possible, to their behavior in the body. Although existing bioreactor designs have cell compartments in which cells can be three-dimensional, the bioreactor designs are flawed in how they supply nutrients and gases to the cells or how they manage cellular waste exchange or secretion of specialized cell products. The supply lines for the bioreactors make use of small, hollow tubes called hollow fibers that are prepared from a liquid that is pressed through sieves into an environment that yields a solid, hollow tube that can be made porous. The pore sizes are typically 0.1-0.7 microns. The pores in these hollow fibers quickly become clogged with material secreted by the cells when cells are placed in the bioreactor. The clogging results in an inability of the cells to survive and function in the bioreactors for very long. There is a loss of specialized function within 7 days for normal cells and a loss of viability within 21 days for normal cells and within 60 days for even highly malignant cancer cells. The invention disclosed herein permits the cells to survive and function indefinitely in the bioreactors. For a preferred embodiment of a bioreactor, see co-pending application Ser. No. 09/586,981 entitled “Bioreactor Design and Process for Engineering Tissue from Cells, with a priority filing date of June 3, 1999, incorporated herein by reference in its entirety.

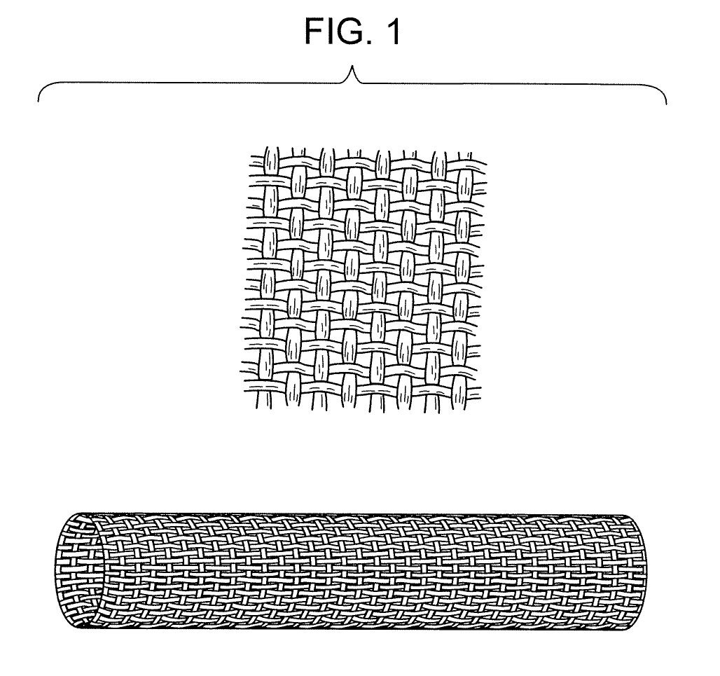

The present invention provides a means to grow healthy liver stem cell based tissues. These tissues can then be used as a bypass or an implant for patients with malfunctioning or failed livers. The use of vascular tubes constructed from fabrics, rather than the fibers obtained from extrusion technologies, provides the means for solving the membrane-fouling problem of Bioartifical Livers. Of the established vascular tubes, woven polyester materials are best because weaves as opposed to knits or braids can have their porosity easily modified and characterized, and polyester has sufficient mechanical patency due to its relatively high integrity and stability to most environments. FIG. 1 illustrates a preferred embodiment of woven vasculatures shown in flat and cylindrical forms. The general methods for the fabrication of such implants are set forth by Gupta et al., “Bio-mechanics of human carotid artery and design of novel hybrid textile compliant vascular grafts,” J. Biomed. Mat. Res. 34:341-349 (1997) and Mizelle et al., “Development of Biomechanically Compliant Arterial Grafts,” Proc. 15^{th} South. Biomed. Eng. Conf., IEEE, 110-113, (1996), each incorporated herein by reference in its respective entirety). Further, the use of vascular tubes made from woven fabrics that are composed of biodegradable materials or natural polymers results in a controlled increase in porosity and selective cell attachment focal points, respectively. The porosity can be modified by varying the spacing and the structure of the yarns in the weave, and the cylindrical shape and rigidity can be established by heat setting woven materials in the desired configuration under optimum conditions of temperature, pressure and residence time. In a preferred embodiment, the biodegradable material is extruded into fibers of high mechanical integrity and then used as a yarn for weaving into the desired vasculature.

Thus, bioreactors and cell compartments are set forth which make use of woven textile vasculatures. The woven textile vasculature is used as a hollow fibrous structure in hollow fiber bioreactors, as a cell surface for flatbed bioreactors, or as bags or tubes for three-dimensional culture systems, for use in expansion and maintenance of cells. The woven textile vasculature can be prepared from any fiber or combination of fiber chemistries such as polyester, polyolefin, cellulose, elastomer, biodegradable fibers, etc. and with any weave design desired. The weave design and the chemistry of the fibers can be adjusted to provide the requisite permeability/porosity of the hollow fibrous structures for engineering of tissues.

### Bioreactor

The instant invention includes a modular multi-coaxial bioreactor, having in theory, no limit to the number of coaxial vasculatures. In a preferred embodiment a scaled-up multi-coaxial bioreactor comprises at least two sets of manifolds, at least three hollow vasculature sizes, at least two sets of endcaps, and a housing. This embodiment of the bioreactor contains at least four separated compartments. The modular design is composed of two sets of manifolds, with each pair of manifolds connected to each end of the vasculatures. There is a series of about 20 to about 400 holes coaxially arranged across the sets of manifolds and coaxially aligning the vasculatures. The manifolds optionally include flow distributors so that fluid and gas phase flow rates through the vasculatures are approximately uniform. The vasculature manifold assemblies are attached radially from the largest to the smallest diameter vasculatures, and axially from the smallest to the largest diameter vasculatures. Vasculatures with smaller diameter are inserted into vasculatures of larger diameter and the respective manifolds are sealed together.

The bioreactors of the current invention advantageously combine ‘integral’ oxygenation with defined diffusion distances, have ports to accommodate potential bile duct formation, and/or are easily scalable. Integral oxygenation permits efficient mass transfer of dissolved gases and control of pH. Defined diffusion distances permit predictable axial and radial physico-chemico-biological parameters such as shear forces, availability of nutrients, and pH. In use with patients, one or more of the at least four compartments can be used for patient blood plasma while another can be used to perfuse cells with integrally oxygenated media. Optionally, two or more bioreactor units are attachable in series so that toxins can perfuse out of plasma radially through the cell mass in one unit and infuse synthetic factors in the next unit. There is the potential for the biliary system to develop using the ports as the bile duct exit ports.

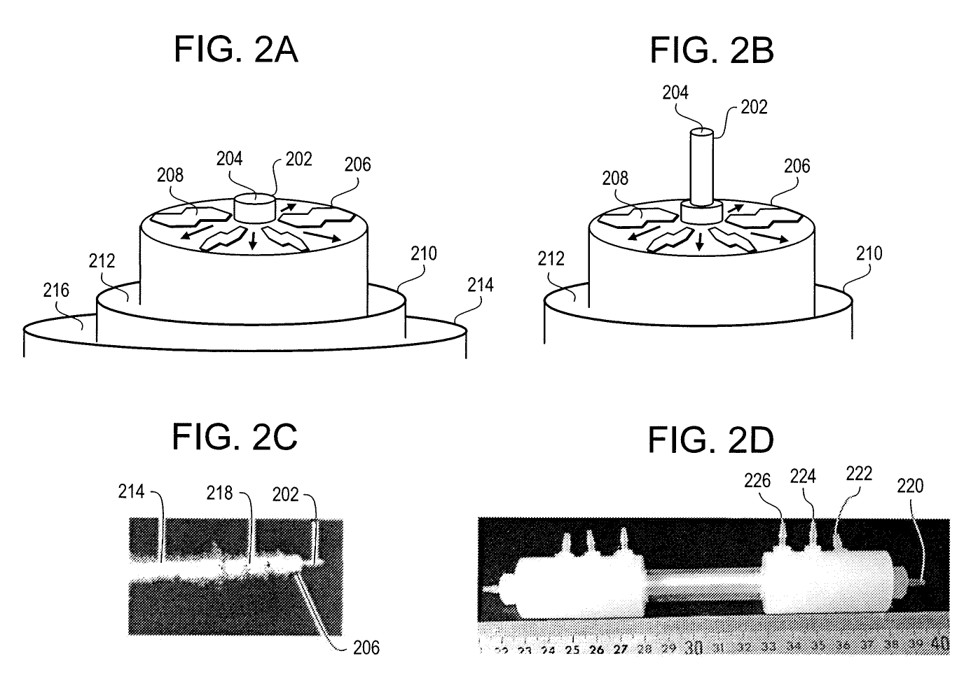

FIG. 2 illustrates two exemplary formats wherein woven cylindrical tubes are incorporated into the multicoaxial bioreactor. FIG. 2A illustrates the air chamber in the outer-most compartment. FIG. 2B illustrates the air chamber in the inner-most compartment.

As shown, FIG. 2A illustrates a multi-coaxial fiber unit according to the instant invention comprising a plurality of compartments. Inner vasculature 202 provides intracapillary space or first compartment 204 for the receipt of standard media or plasma. Middle vasculature 206 provides annular space or first middle compartment 208 for the containment of cells such as liver cells. Outer vasculature 210 provides extracapillary space or second middle compartment 212 for the receipt of media. Housing 214 defines the outermost perimeter of the multi-coaxial fiber unit. Space or outermost compartment 216 between housing 214 and outer vasculature 210 allows for the receipt of a gas.

Similarly, in FIG. 2B inner vasculature 202 provides intracapillary space or first compartment 204 for the receipt of a gas. Middle vasculature 206 provides annular space or first middle compartment 208 for the containment of cells such as liver cells. Outer vasculature 210 provides extracapillary space or second middle compartment 212 for the receipt of media.

FIG. 2C illustrates a photographic view of an embodiment of the woven fabric incorporated into a multi-coaxial bioreactor, with air chamber in the outermost chamber, illustrating inner vasculature 202, middle vasculature 206, housing 214, and aeration fiber 218.

FIG. 2D illustrates openings leading to ports to allow for the movement of materials. Innermost port(s) 220 allow for the flow of media or plasma through the bioreactor. First middle port(s) 222 allow for the inoculation of cells into, or flow of cells through, the bioreactor. Second middle port(s) 224 allow for the flow of media through the bioreactor. Lastly, outermost port(s) 226 allow for the flow of gas through the bioreactor. Alternative uses of ports are also envisioned. For example, media can flow through port(s) 226, cells into, or through, port(s) 224, media or plasma through 222, and oxygen or other gases through 220.

### Identification of Optimum Basic Vasculature for BAL Bioreactor

Property-structure correlation and hydraulic permeability-tissue growth study are used to identify the specifications that provide an ideal stable vasculature for bioartificial liver application(s) and the technological/structural settings that produce such vasculatures on a consistent basis. Several different polyester yarns, differing in linear density and number of filaments are used. Vasculatures of a number of different tightnesses are woven from each yarn. Vasculatures of two different diameters, for use as co-axial bioreactors, are woven. The heat setting conditions that yield the most stable vasculature configuration are identified. The tubes are characterized for porosity, hydrolic permeability, compressional resilience and pore size distribution. Porosity is determined through the use of a structural model relating to the LaPlace equation, which is based on the spacings between the yarns, the diameters of the yarns, and the geometry of the plain woven fabric. Hydrolic permeability is determined experimentally using Darcy's equation. (Darcy's Equation is a formula stating that the flow rate of water through a porous medium is proportional to the hydraulic gradient, and is defined further below.)

Compressional resilience is determined using an Instron tensiometer, equipped with a compression cell. Pore size distribution is determined using a liquid extrusion device and flat specimens having the same specifications as the tubular vasculatures.

Darcy's Equation permits one to estimate the correlation between pressure difference and radial flow given the hydraulic permeabilities of the material under consideration. The model assumes incompressible and Newtonian fluid, that the axial pressure gradient is negligible, and that the flow rate across the vasculatures is constant. Deriving this equation for two concentric hollow vasculatures the following relationship is obtained.

$$Δ P = \frac{Q}{2 π L} [ \frac{ln ( \frac{{r}_{b}}{{r}_{a}} )}{{K}_{1}} - \frac{ln ( \frac{{r}_{d}}{{r}_{c}} )}{{K}_{2}} ] \qquad ( II )$$

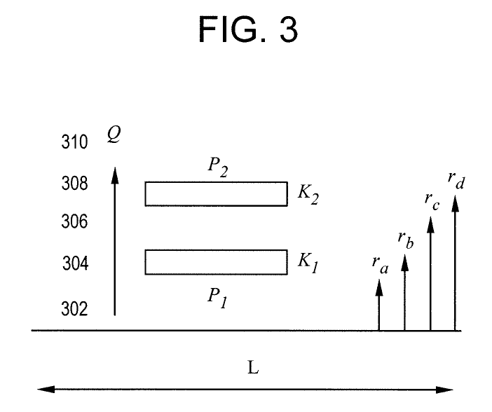

FIG. 3 defines the variables used in the equation. Q is radial flow rate from compartment 302 characterized by a hydrostatic pressure P₁, through pores in fiber 304 characterized by hydraulic permeability K₁, through intermediate compartment 306, then through pores in second fiber 308 characterized by hydraulic permeability K₂ to compartment 310 characterized by hydrostatic pressure P₂.

The values obtained relating to these variables and characterizations are correlated to provide a structure-property correlation model. Thus, data from the bioartificial liver bioreactor study disclosed herein provides a model for selecting optimum specifications for producing the vasculature for use in varying applications, without the need for experimental determinations. These applications include but are not limited to bioreactors, organ assist devices, implantable tissues, grafts, and the like.

### Development of Next Generation Vasculatures for BAL Bioreactor Application

Here, biodegradable and transversely compliant vasculatures are developed. The optimum Basic Vasculature for Bioartificial Liver Bioreactor identified as described above, is used. Biodegradable fibers combined with nonbiodegradable fibers are used as warp and weft elements in construction of tubes. (Warp is the set of fibers that run along the length of the material and weft is the set of fibers that are inserted from the side and cover the width. Warp is wound on a beam and run threaded through a loom. Weft is inserted through warp by lifting and lowering alternative warp threads so that there is interlacing.) The rate at which these degrade and the tissue reaction they cause is examined using standard procedures. A polymer is selected and combined with polyester in novel ways for the construction of grafts. The amount of biodegradable fiber used relative to non-biodegradable provides the means for setting the initial and final limits of porosity for the vasculature.

A second variant is the development of vasculatures with an elastomer combined with polyester for use as weft yarn. The amount and type is varied in order to get different degrees of transverse stretchabilities and, thus, transverse compliances. The level of transverse compliance can be characterized on a specially equipped Instron tensiometer.

### Optimization of Hydraulic Permeability and Flow Configuration

As disclosed herein, in a preferred embodiment, liver progenitors are expanded on biodegradable microcarriers in the space between the two coaxial fibers to generate the entire liver maturation lineage. Thus, the loading density of the progenitors per fiber pair must be minimized to optimize the number of bioartificial livers per human donor. This requires the resolution of two engineering problems. First, the optimum hydraulic permeability of the two coaxial vasculatures sandwiching the cell mass must be determined. Second, the optimum flow configuration to minimize or compensate for membrane fouling and corresponding decrease in hydraulic permeability with cell growth must be determined. In a preferred embodiment, the hydraulic permeability values of the two fibers are similar, such that a peristaltic type of flow configuration can be used to maintain clean nutrient and waste paths.

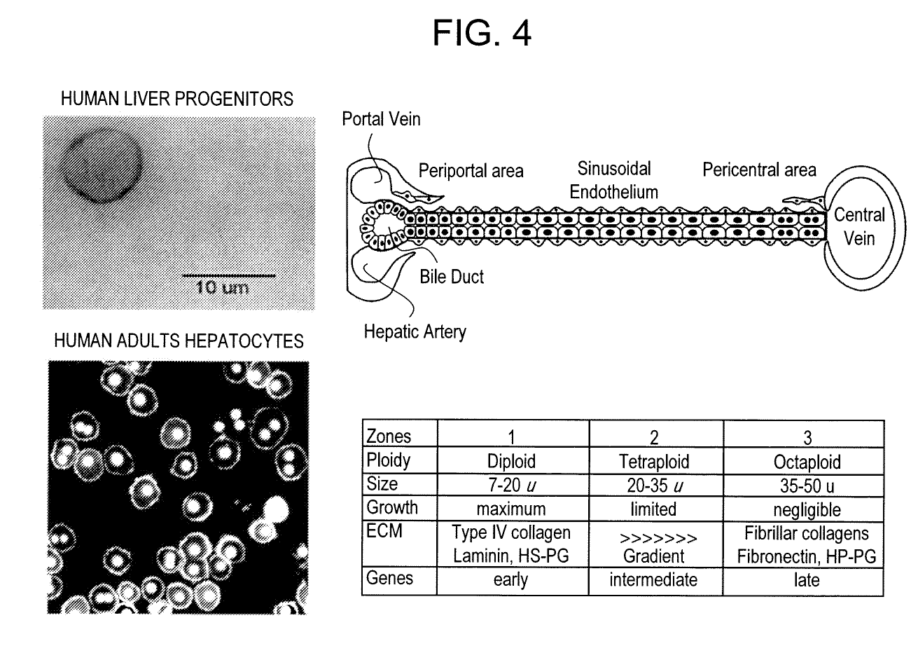

FIG. 4 illustrates a liver lineage model. In a preferred embodiment, progenitors or stem cells feed the lineage of the bioreactor in the same fashion as in the liver acinus. Thus an architecture is provided similar to that used in the liver acinus, wherein progenitors are used to seed the bioreactor and with the correct flow of blood, will result in maturation similar to that which occurs in the liver.

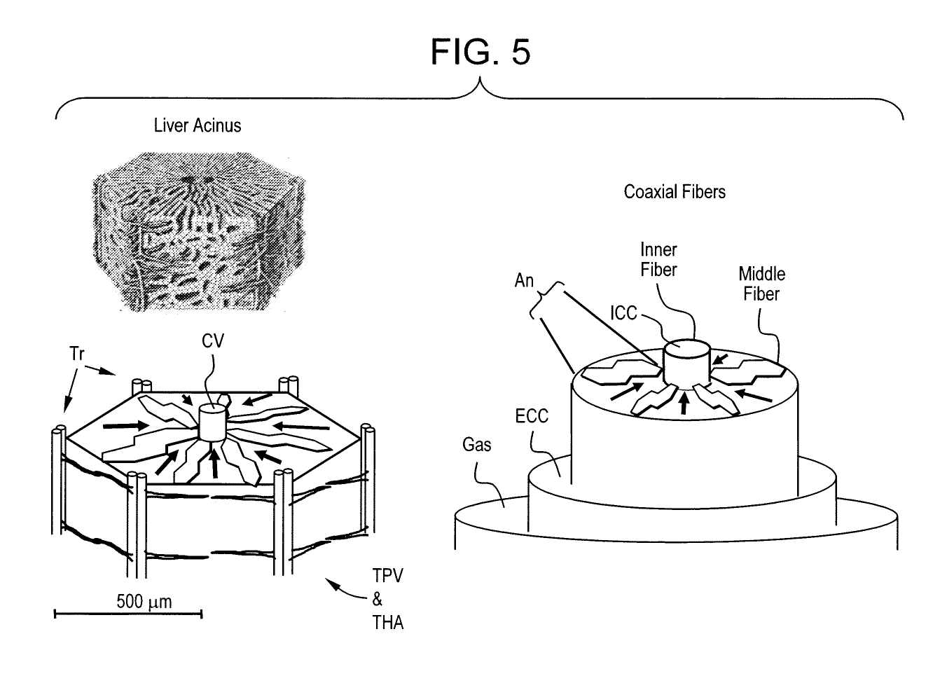

FIG. 5 illustrates a multicoaxial bioreactor design. Through the use of this design a preferred flow is achieved.

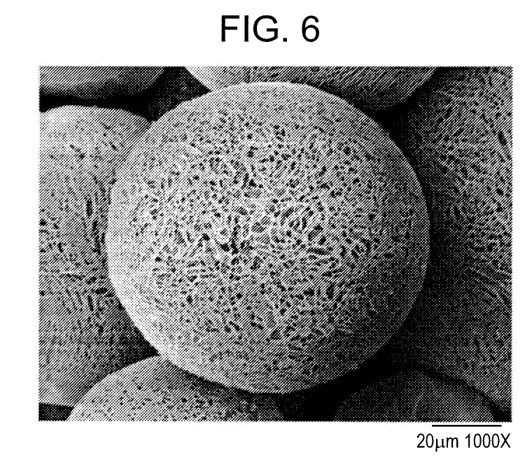

FIG. 6 illustrates porous, biocompatible, biodegradable polylactide glycolic acid (PLGA) microcarriers for cells in bioreactors. In a preferred embodiment, the progenitors referred to in FIG. 4, above, are seeded onto these PLGA microcarriers/beads.

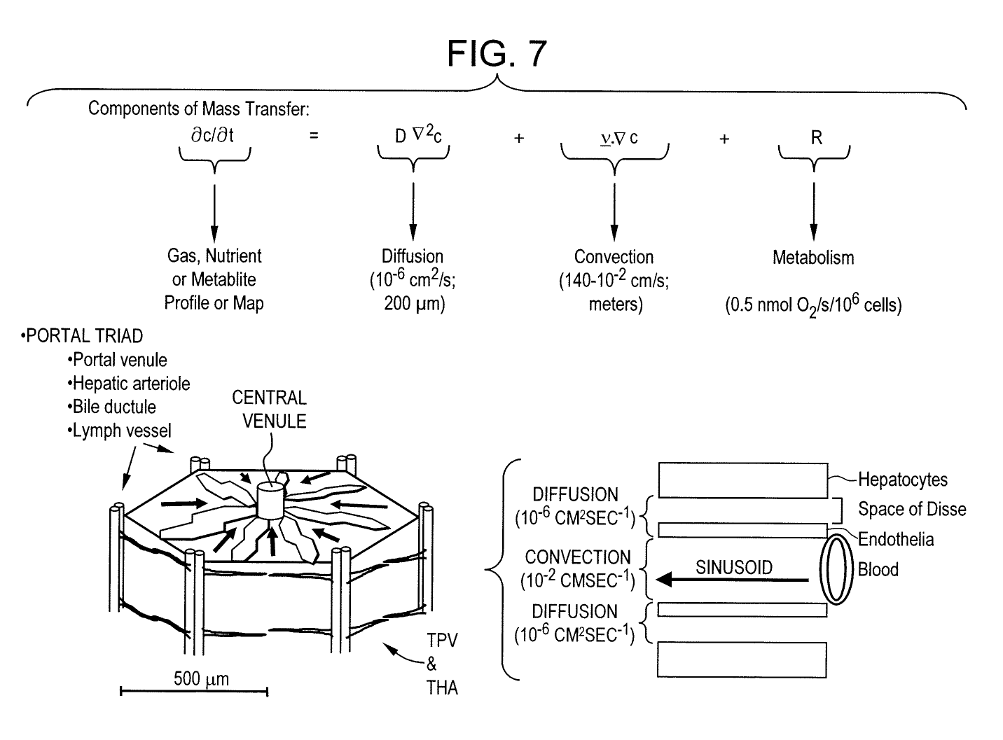

FIG. 7 illustrates a physical analysis of the liver acinus, providing an illustration of Darcy's law. Due to the large distance, diffusion alone cannot provide needed oxygen. Thus, mass transfer is dependent on convention and pressure differentials.

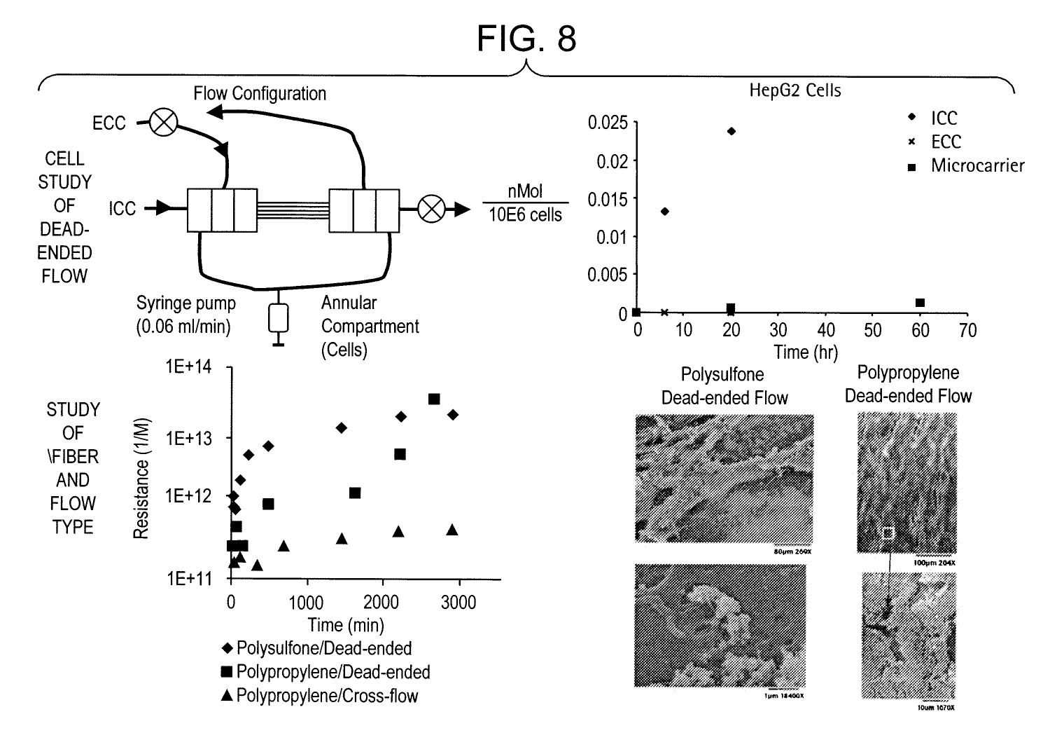

FIG. 8 illustrates membrane fouling studies. As shown, pores in the polypropylene fibers clog quite rapidly causing an increase in pressure and cell death.

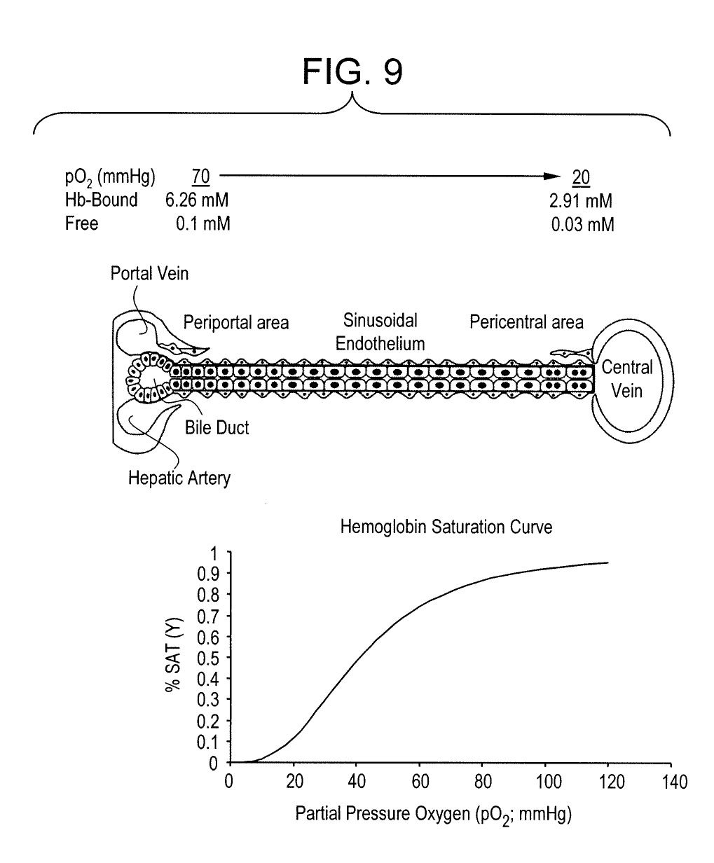

FIG. 9 illustrates the effect of no hemoglobin on oxygen mass transfer. This figure illustrates hemoglobin's efficiency in providing oxygen. It also augments the fact that hemoglobin is the preferred oxygen carrier, and that one cannot depend upon diffusion to oxygenate, particularly when the carrier is water. However, due to the velocity used in the preferred embodiment the drop is not as great.

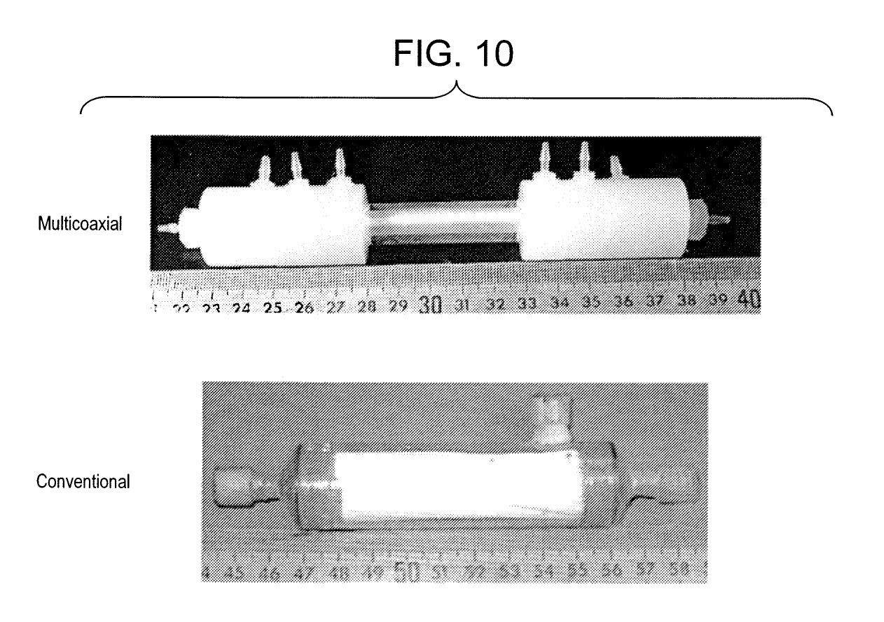

FIG. 10 illustrates a comparison of a conventional, with a multicoaxial, bioreactor.

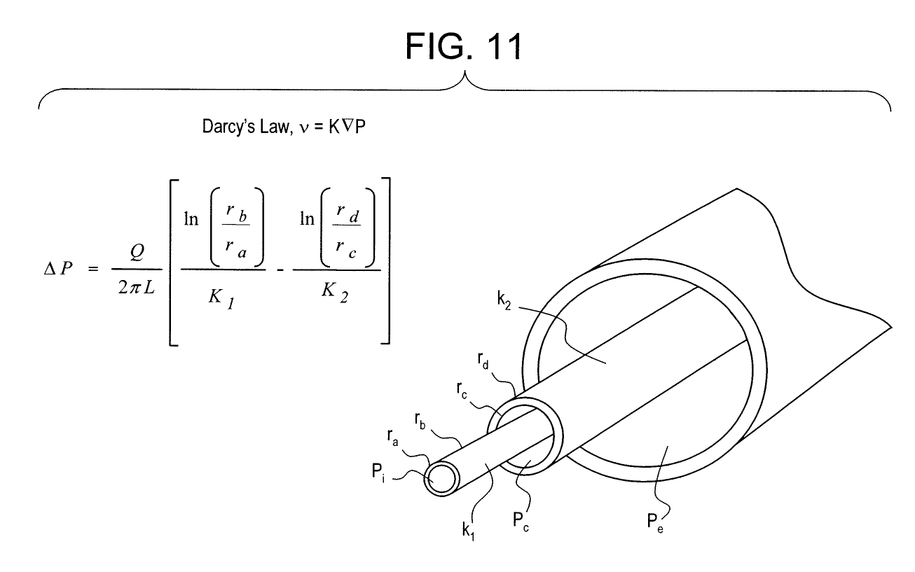

FIG. 11 illustrates a hydrodynamic model, providing an application of Darcy's law.

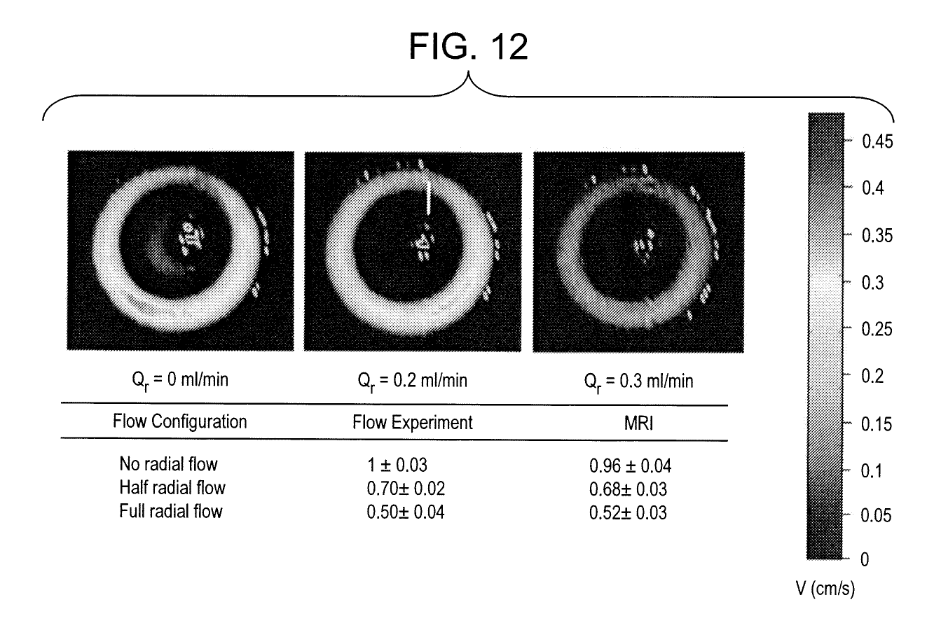

FIG. 12 illustrates the use of MRI to determine axial flow.

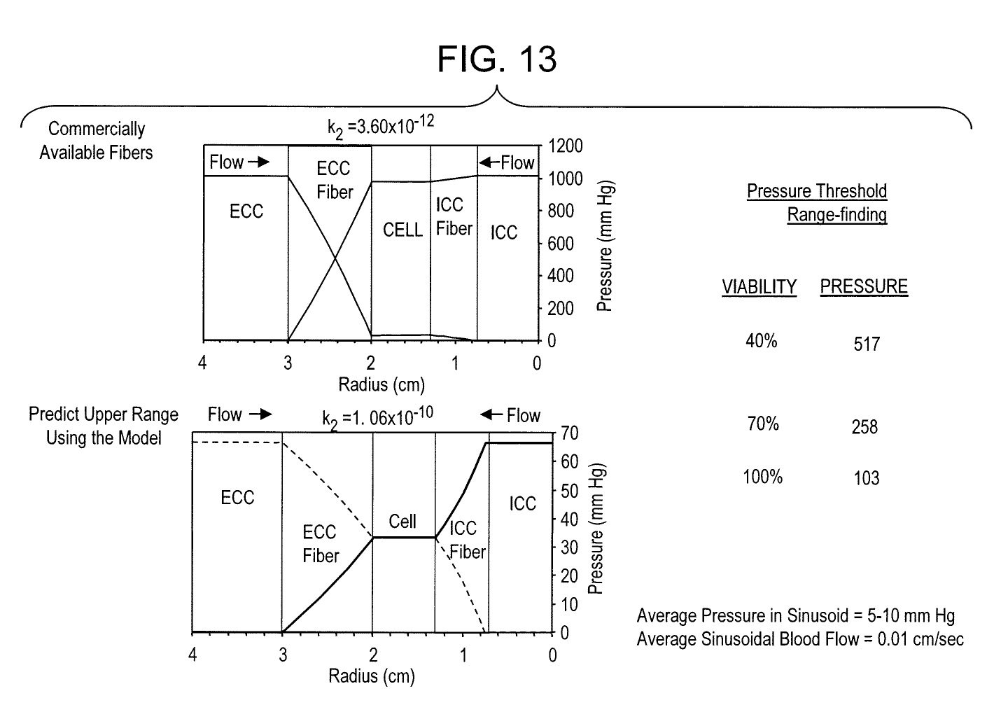

FIG. 13 illustrates predicted pressure profile and optimum K₁ and K₂. As shown, 100 percent viability is obtained with a pressure of 103 mm Hg. At a pressure of 517 mm Hg the viability reduces to 40 percent. the average pressure in sinusoid is about 5 to 10 mm Hg. While the average sinusoidal blood flow is 0.01 cm/sec.

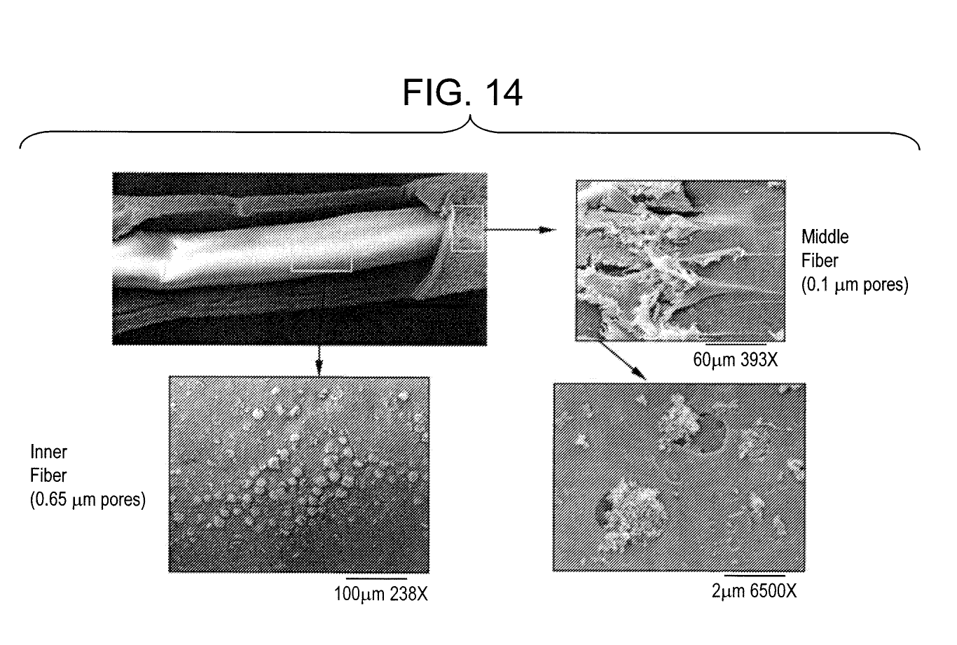

FIG. 14 provides photographic illustrations of membrane fouling and its adverse effect on mass transfer. As stated, membrane fouling causes pressure increase and cell death.

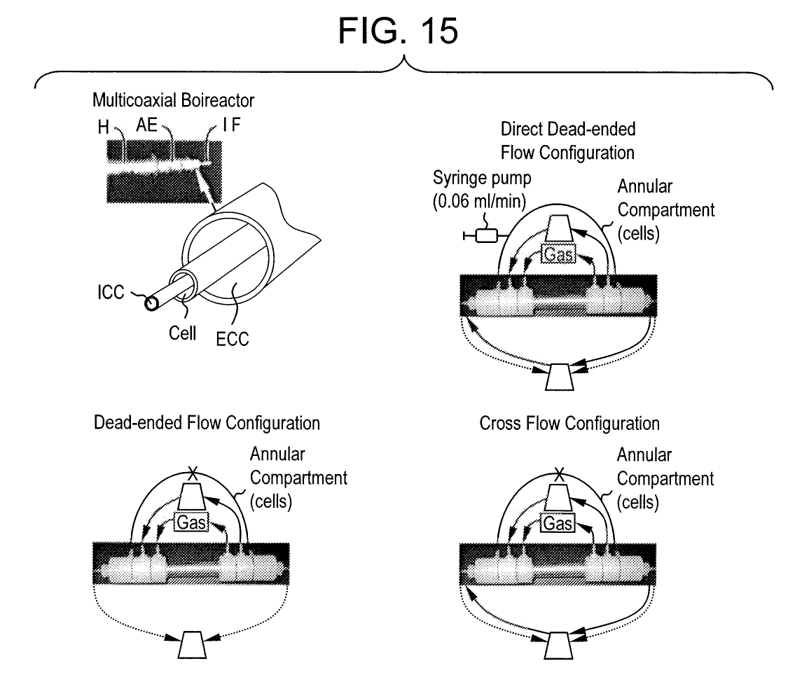

FIG. 15 illustrates dead-end and cross flow configurations used for the fouling study.

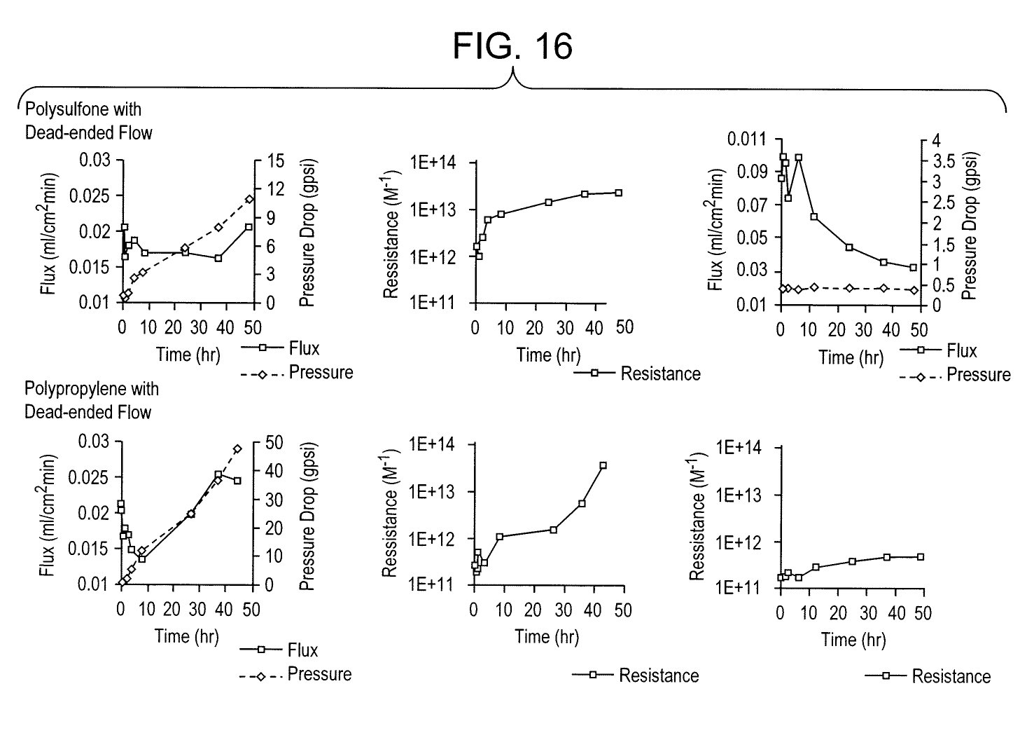

FIG. 16 provides results of dead-end and cross flow configurations for fouling study.

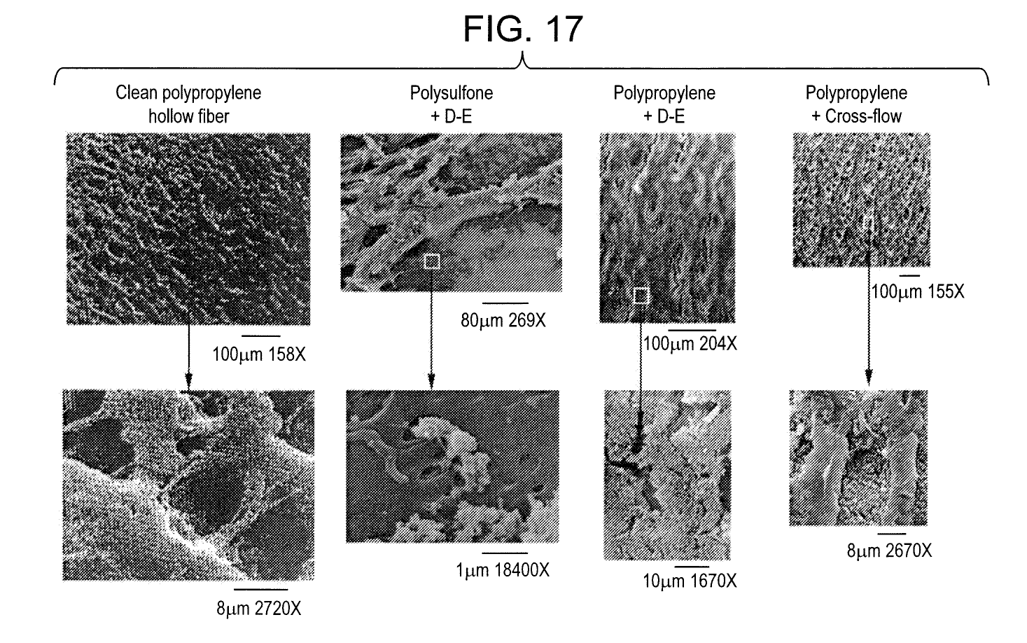

FIG. 17 provides photographic results of dead-end and cross flow configurations for fouling study.

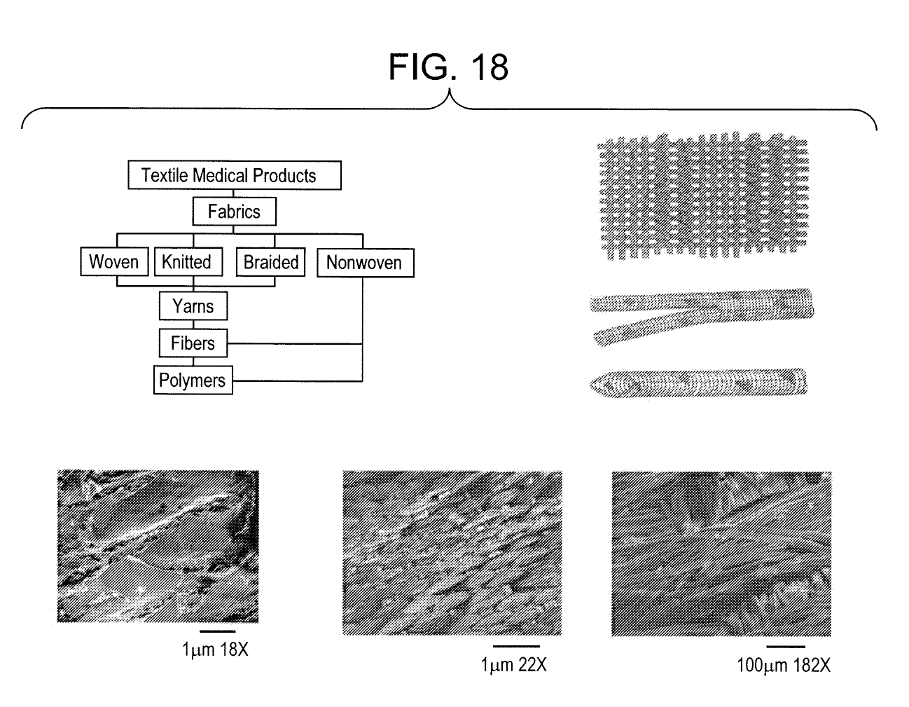

FIG. 18 provides photographic results of fouling studies of woven vasculature incorporated into multicoaxial bioreactors.

The bioreactors of the current invention advantageously combine ‘integral’ oxygenation with defined diffusion distances, have ports to accommodate potential bile duct formation, and/or are easily scalable. Integral oxygenation permits efficient mass transfer of dissolved gases and control of pH. Defined diffusion distances permit predictable axial and radial physico-chemico-biological parameters such as shear forces, availability of nutrients, and pH. In use with patients, one or more of the compartments can be used for patient blood plasma while another can be used to perfuse cells with integrally oxygenated media. Optionally, two or more bioreactor units are attachable in series so that toxins can perfuse out of plasma radially through the cell mass in one unit and infuse synthetic factors in the next unit. There is the potential for the biliary system to develop using the ports as the bile duct exit ports.

### EXAMPLES

The following specific examples are provided to better assist the reader in the various aspects of practicing the present invention. As these specific examples are merely illustrative, nothing in the following descriptions should be construed as limiting the invention in any way. Such limitations are, or course, defined solely by the accompanying claims.
1) NMR Analysis of Liver Cell Function in the One-Sided Multi-Coaxial Hollow Fiber Bioreactor.

Sprague-Dawley rats are anesthetized with pentobarbital (50 mg/kg intraperitoneally). The liver is exposed by a ventral midline incision and the portal vein is cannulated for infusion of cell dissociation solutions. The liver cells are dissociated by sequential infusions of ethylene diamine tetraacetic acid (50 mM) and collagenase (1 to 20 mg/ ml) in Krebs-Henseleit buffer, pH 7.4. Adequate perfusion of the liver is indicated by uniform blanching of the liver. Isolated cells are collected and introduced into the cell compartment of the one-sided multi-coaxial hollow fiber bioreactor.

Nuclear magnetic resonance (NMR) is performed using an NMR probe design composed of two Helmholtz coils photo-etched onto flexible copper-coated composite. The two coils, suitably insulated, are wrapped around the bioreactor and oriented orthogonally to each other. The inner coil is tuned to 81 MHz for study of energy metabolism as measured by changes in the spectrum of ³¹P. The probe and bioreactor assembly is placed on a centering cradle in the isocenter of the magnet for optimal comparison of spectra. The aerated nutrient medium is supplied to the first compartment inlet port of the bioreactor. Integral aeration is provided by flow of a 95% air with 5% CO2 mix through inlet port 4, associated with the outermost or fourth compartment of the bioreactor. Ham's F-12 nutrient medium is pumped through compartment 3 with a peristaltic pump. The temperature of the reservoir of medium is maintained at 42° C. with a temperature controlled water bath, so as to maintain the bioreactor temperature at 37° C. The NMR signal from γ-31P nucleotide triphosphates and B-³¹P nucleotide diphosphates, other cellular components of energy metabolism, and biosynthesis are analyzed. The NMR signal is monitored as a function of mass transfer dictated by gas flow rate and oxygen percentage, nutrient medium flow rates, and cell loading densities.
2) Oxygen Flux in the Absence of Cells.

Oxygen microelectrodes are connected to a transducer and Workbench™ software, and then calibrated against known standards. The calibrated oxygen microelectrodes are placed at intervals along the fiber length in the second compartment of the multi coaxial hollow fiber bioreactor. A reservoir of plasma is attached to the inlet port of the first compartment, the innermost compartment of the multi-coaxial hollow fiber bioreactor. A reservoir of RPMI 1640 nutrient medium is attached to the inlet port of the third compartment. Peristaltic pumps are arranged in-line to circulate the plasma and nutrient medium. The second compartment is also filled with nutrient medium. The signal from each microelectrode is acquired at ten-second intervals and processed by the software for conversion to oxygen tensions. The gas phase is switched between 95% air with 5% CO₂ and 95% N₂ with 5% CO₂ at selected intervals. Rates of depletion and recovery of oxygen tension are measured at different flow rates to evaluate oxygen flux in the absence and presence of cells.
3) Use as an Extracorporeal Liver Assist Device for Evaluation of Bilirubin.

The Gunn rat model, (the animal model for Crigler Naijar syndrome in humans) is an ideal model for demonstrating the efficacy of the bioreactor as an extracorporeal liver assist device. The Gunn rat has a defect inherited as an autosomal recessive trait in Wistar rats. The defect, present in homozygous recessive animals, is in the gene encoding UDP glucuronosyltransferase, an enzyme necessary for the conjugation and biliary excretion of bilirubin (a breakdown product of hemoglobin in senescent red blood cells). The Gunn rat therefore cannot conjugate and excrete bilirubin and becomes hyperbilirubinemic, having serum bilirubin levels of about 5-20 mg/dL, compared with 1 mg/dL in normal rats.

A scaled-up multi-coaxial hollow fiber bioreactor is used as an extracorporeal liver assist device with Gunn rats. The livers of heterozygous (phenotypically normal) Gunn rats are perfused and the cells are isolated. The cells are suspended in Dulbecco's Modified Eagle Medium (DMEM) and 10⁹ cells are introduced into the second compartment of the bioreactor. Blood from the femoral artery of a Gunn rat (total average blood volume ca. 10 to 12 mL) is perfused through the third compartment of the bioreactor, separated from the liver cell annular space by the wall of the hollow fiber, at a flow rate of about 0.6-0.8 mL/min with the aid of a peristaltic pump. At the same time, DMEM is flowed through the compartment one of the bioreactor at a flow rate of about 0.5 mL/min. Blood flowing out of the bioreactor is returned to the Gunn rat.

The levels of unconjugated and conjugated bilirubin in blood exiting the bioreactor are determined over the course of six hours using the Sigma Total and Direct Bilirubin assay system according to the instruction supplied by Sigma Chemical Company (Sigma Procedure #522/553).
4) Biosynthetic Hepatocyte Function in a Scaled-Up Multi-Coaxial Hollow Fiber Bioreactor/BAL.

Isolated liver cells are further separated by zonal centrifugation in sucrose density gradients. Density fractions corresponding to parenchymal cells are collected and introduced into the aseptic cell compartment (compartment 2) of the scaled-up multi-coaxial bioreactor.

The parenchymal cells are maintained by circulating warm Ham's F-12 nutrient medium through compartments 1 and 3, and 95% air with 5% CO₂ through the fourth compartment. The effluent from the first compartment is collected and fractions are analyzed for parameters of biosynthetic liver function. Albumin synthesis is measured by enzyme-linked immunosorbent assay.
5) Biotransformatory Function in a Scaled-Up Multi-Coaxial Hollow Fiber Bioreactor/BAL.

Isolated liver cells are further separated by zonal centrifugation in sucrose density gradients. Density fractions corresponding to Kupffer cells are collected and introduced into the second compartment (cell compartment) of the scaled-up multi-coaxial hollow fiber bioreactor.

The cells in the bioreactor are maintained by circulating DMEM (without Phenol Red) through the inlet and outlet ports for the first and third compartments and 95% air with 5% CO2 through the ports for the fourth compartment. The cells are permitted to adhere within the compartment, followed by the introduction of free hemoglobin (1-10 mg/ml) into the first compartment. The appearance of hemoglobin and the metabolic products of hemoglobin in the third compartment are monitored with an in-line spectrophotometer.
6) The Serially-Linked Bioreactor with Human Cells for Patient Treatment.

Human hepatoma C3A cells are cultured as described (Mickelson, J. K. et al. Hepatology 1995, 22, 866) and introduced into all the second compartments of the serially-linked bioreactor. Nutrient medium and 95% air with 5% CO₂ are pumped through the third and outermost compartments, respectively, and cell growth is monitored by glucose utilization. When the cells have attained the plateau, or stationary, growth phase, the albumin output is monitored.

The blood of a patient suffering liver failure is separated into plasma and cells by plasmapheresis and the plasma is pumped into the first compartment of the first bioartificial liver subunit. A portion of the plasma flows radially from the first compartment through the cell compartment to the third compartment to form biotransformed effluent. The plasma exits the first compartment of the first bioartificial liver subunit and flows into the third compartment of the second bioartificial liver subunit. The biotransformed effluent from the third compartment of the first bioartificial liver subunit and flows into the first compartment of the second bioartificial subunit. Radial flow in the first bioartificial liver subunit detoxifies a portion of the plasma and radial flow in the second bioartificial liver subunit contributes biosynthetic products to the plasma to form supplemented plasma. Vital signs, jaundice, and blood level of toxins are monitored at regular intervals. Flow rates of plasma and medium are adjusted to maximize biotransformation of circulating toxins. Survival of the patient is measured.
7) Extracellular Matrix Effects on Differentiation of Hepatocytes in the Scaled-Up Multi-Coaxial Hollow Fiber Bioreactor.

Parenchymal cells are isolated by zonal centrifugation, suspended in reconstituted basement matrix from the Englebreth-Holm-Swarm mouse sarcoma, and introduced into the second compartment (cell compartment) of the scaled-up multi-coaxial bioreactor. The hepatocytes are arrested in a G₀ state by adhesion to the basement matrix, and are maintained in the normal hepatic phenotype. The highly differentiated state is characterized by synthesis of albumin and hepatic transcription factors such as C/EBP-. The parenchymal cells are maintained by circulating warm Ham's F-12 nutrient medium through the first and third compartments, and 95% air with 5% CO₂ through the fourth compartment. The effluent from the first compartment is collected and fractions are analyzed for parameters of biosynthetic liver function. Albumin synthesis is measured by enzyme-linked immunosorbent assay.
8) Growth and Differentiation of Human Hepatocytes in the Scaled-Up Multi-Coaxial Hollow Fiber Bioreactor.

Human parenchymal hepatocytes are isolated by the method of (Block, G. D. et al. J Cell Biol 1996, 132, 1133) and introduced into the second compartment of the scaled-up multi coaxial hollow fiber bioreactor. The parenchymal cells are propagated by exposure to hepatocyte growth factor (HGF/SF), epidermal factor, and transforming growth factor alpha in nutrient medium HGM introduced into the third compartment and air:CO₂ (19:1) introduced into the fourth compartment. The ratio of transcription factor C/EBP to C/EBP is decreased by this process and the cell synthesis of albumin also is decreased. The medium flowing through the third compartment is modified to include transforming growth factor and epidermal growth factor to induce differentiation of the cells and synthesis of albumin, in the formulation described (Sanchez, A. et al. Exp Cell Res 1998, 242, 27).
9) Biosynthesis of Hormones and Factors in the Scaled-Up Multi-Coaxial Hollow Fiber Bioreactor.

Parathyroid glands are obtained aseptically, minced, and treated with collagenase as described (Hornicek, F. L. et al. Bone Miner 1988, 4, 157). The dispersed cells are suspended in CMRL-1415 nutrient medium supplemented with fetal bovine serum and introduced into the second compartment of the scaled-up multi-coaxial bioreactor. A mixture of 95% air with 5% CO₂ is pumped through the fourth port. Warm medium is pumped through the first and third ports and the effluent from the chamber is concentrated by ultrafiltration for collection of parathyroid hormone, parathyroid hypertensive factor, and other cell products. The hormones and factors are purified by immunoprecipitation and chromatography.
10) The Five Compartment Serially-Linked Bioreactor with Human Cells for Patient Treatment.

Human hepatoma C3A cells are grown as in example VI, above, except in the third compartment of a five-compartment serially-linked bioreactor. The innermost compartment (compartment 1) and the outermost compartment (compartment 5) are suffused with the gas mix, 95% air with 5% CO₂. Nutrient medium is pumped through the second and fourth compartments, respectively, and cell growth is monitored by glucose utilization. When the cells have attained the plateau, or stationary, growth phase, the albumin output is monitored.

The blood of a patient suffering liver failure is separated into plasma and cells by plasmapheresis and the plasma is pumped through the serially connected second compartments of the bioreactor. Vital signs, jaundice, and blood level of toxins are monitored at regular intervals. Flow rates of plasma and medium are adjusted to maximize biotransformation of circulating toxins. Survival of the patient is measured.

Various publications have been referred to throughout this application. The disclosures of these publications in their entireties are hereby incorporated by reference into this application to more fully describe the state of the art to which this invention pertains.

The purpose of the above description and examples is to illustrate some embodiments of the present invention without implying any limitation. It will be apparent to those of skill in the art, in light of this teaching, that various modifications and variations may be made to the composition and methods in the present invention to generate additional embodiments without departing from the spirit or scope of the invention. The specific composition of the various elements of the bioreactor system, for example, should not be construed as a limiting factor. Accordingly, it is to be understood that the drawings and descriptions in this disclosure are proffered to facilitate comprehension of the invention and should not be construed to limit the scope thereof.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. Thus, the invention is properly limited solely by the claims that follow.

What is claimed is:

1. A device for maintaining viable eucaryotic cells, comprising:
(a) woven fabric forming an annular compartment, having an annular space, wherein the annular space comprises a biodegradable polymeric material,
(b) at least two additional compartments, adjacent and coaxial to said annular space, where each adjacent compartment contains a liquid, and
(c) an integral aeration supply for the annular space.

2. The device of claim 1, wherein the woven fabric comprises polyester.

3. The device of claim 1, wherein the biodegradable material comprises collagen.

4. A bioreactor, comprising:
(a) a housing having an inner side comprising: a gas introduction means integral to the housing; and a gas expiration means integral to the housing;
(b) an array of a plurality of modules of textile vasculatures, residing within the housing, each module comprising:
(i) a plurality of coaxial textile vasculatures, each having an inner side and an outer side, including an innermost textile vasculature and an outermost textile vasculature;
(ii) a plurality of compartments, comprising: a first compartment defined by the inner side of the innermost textile vasculature; and
(iii) at least one additional compartment defined by a respective annular space between adjacent fibers of the plurality of coaxial textile vasculatures; and
(c) an outermost compartment defined by a space within the inner side of the housing which is not occupied by the plurality of modules,
wherein the at least one additional compartment comprises a biodegradable polymeric material.

5. The bioreactor of claim 4, wherein the biodegradable material comprises collagen.

6. A serially-linked bioreactor, comprising a plurality of bioreactor subunits, each bioreactor subunit comprising:
(a) a housing having an inner side and an outer side, said inner side comprising: a gas introduction means integral to the housing; and a gas expiration means integral to the housing;
(b) an array of a plurality of modules of textile vasculatures, residing within the housing, each module comprising:
(i) a plurality of coaxial textile vasculatures, each having an inner side and an outer side, including an innermost textile vasculature and an outermost textile vasculature;
(ii) a plurality of compartments, comprising: a first compartment defined by the inner side of the innermost textile vasculature; and at least one additional compartment defined by a respective annular space between adjacent vasculatures of the plurality of coaxial textile vasculatures; and
(c) an outermost compartment defined by a space within the inner side of the housing which is not occupied by the plurality of modules; and
(d) at least one compartment of one bioreactor subunit linked serially to at least one compartment of at least one other bioreactor subunit,
wherein the at least one additional compartment comprises a biodegradable polymeric material.
